(19) United States
(12) Patent Application Publication
(10) Pub. No.: US 2026/0097230 A1
(43) Pub. Date: Apr. 9, 2026

(54) CIRCULAR DICHROISM USING UV LIGHT TO TREAT CANCER AND METHOD THEREOF

(71) Applicant: Kurt A. Garrett, Raleigh, NC (US)

(72) Inventor: Kurt A. Garrett, Raleigh, NC (US)

(21) Appl. No.: 18/907,002

(22) Filed: Oct. 4, 2024

(51) Int. Cl. A61N 5/06

(52) U.S. Cl. CPC A61N 5/0613; A61N 2005/0661; A61N 2005/0667

(57) ABSTRACT

A method of treating cancer using UV light delivered directly to a tumor via circular dichroism.

### COPYRIGHT NOTICE

A portion of the disclosure of this patent contains material that is subject to copyright protection. The copyright owner has no objection to the reproduction by anyone of the patent document or the patent disclosure as it appears in the Patent and Trademark Office patent files or records, but otherwise reserves all copyright rights whatsoever.

### BACKGROUND OF THE INVENTION

### Field of the Invention

The present invention relates to a method of treating cancer in a mammal. In particular, it relates to circular dichroism using UV (ultraviolet) light for treating cancer.

### Description of Related Art

Cancer tumors evade destruction by a number of different mechanisms, including multiple regulatory pathways and cells types.

A number of cells play a role in the containment of cancer, including tumor infiltrating lymphocytes, regulatory T cells, CD8⁺ cytotoxic T cells, CD4⁺ helper T cells, myeloid-derived suppressor cells, immune inhibitory B cells, tumor associated macrophages, natural killer cells, dendritic cells, and antigen presenting cells (APCs). Tumor cells may reprogram myeloid cells to create an immunosuppressive tumor environment and drive tumor progression. For example, factors associated with tumor immunosuppression include tumor growth factor-β and interleukin (IL)-10, as well as ligands that down modulate tumor infiltrating lymphocyte activity, such as programmed death ligand (PD-L1). As such, an approach to the treatment of cancer is based on immune checkpoint blockade therapy, such as antibodies that block cytotoxic T lymphocyte antigen 4 (CTLA-4) and PD-1, chimeric antigen receptor (CAR) T cell immunotherapy, and therapeutic vaccines, such as T cell and dendritic cell vaccines with or without tumor antigens or tumor neoantigens.

Based on the recognition of the role of the immune system in controlling tumorigenesis and tumor progression, a number of cancer immunotherapeutic agents, including check point inhibitors, targeting immune tolerance and combinations thereof have been or are currently being investigated. Most cancer immunotherapeutic agents are categorized as 1) drugs that target tumor immune evasion via blockade of negative regulatory signals (e.g., co-inhibitory checkpoints and tolerogenic enzymes); and 2) drugs that directly stimulate immunogenic pathways (e.g., agonists or co-stimulatory receptors), or a combination of co-inhibitory and co-stimulatory agents. Other immunostimulatory strategies include enhancers of antigen presentation (e.g., dendritic and T cell vaccines), the use of exogenous recombinant cytokines, oncolytic viruses, and cell therapies using native or modified antigen-competent immune cells.

### Drugs that Target Immune Tolerance Via Checkpoint Inhibitors

Co-inhibitory agents include, but are not limited to, antibodies against CTLA-4 and PD-1, antagonist of T cell immunoglobulin and mucin domain (TIM-3), antagonist of v-domain immunoglobulin-containing suppressor of T cell activation (VISTA, also known as PD-1 homolog), antagonist of lymphocyte activation gene 3 (LAG-3), drugs that target T cell immunoglobulin (TGIT), TGFβ receptor type 1 inhibitor, and CD19 and CD3 bispecific antibodies. Other strategies that target checkpoint inhibitors include adaptive resistance to checkpoint inhibition.

### Drugs that Enhance Antitumor Responses, Co-Stimulatory Agents

Co-stimulatory agents include, but are not limited to, cancer vaccines, either whole cell or specific peptide antigen preparations, e.g., tumor specific antigens or tumor associated antigens. Antigen presenting cells, such as macrophages or dendritic cells, display these tumor antigens to activate B and T cells. In general, tumor vaccines are administered with an adjuvant nonspecific immune stimulant. For example, granulocyte macrophage colony stimulating factor (GM-CSF) may be administered with a dendritic vaccine to increase the immune response to the tumor. Vaccines, including dendritic cell vaccines may be treated with a tumor specific antigen. Tumor specific antigens are highly tumor specific and expressed only on tumor cells. Tumor associated antigens are more widely expressed in both tumor and nontumor cells. Most vaccine peptide targets are restricted to specific HLA haplotypes, however, and may include somatic mutations of cancer antigens (i.e., tumor neoantigens). Molecules, such as heat shock proteins may serve as a chaperone for tumor peptides.

Antigen-presenting cells may be obtained from any source, such as peripheral blood mononuclear cells, peripheral blood monocytes, circulating stem cells, stem cells or precursor cells derived from bone marrow, peripheral blood, cord blood; or antigen-presenting cells may be found in tissue parenchyma, generated in vitro, obtained from a commercial source or cloned. Antigen-presenting cells include, but are not limited to, monocytes, macrophages, dendritic cells, Langerhans cells, lymphocytes, hematopoetic stem cells, peripheral blood stem cells, peripheral blood mononuclear cells, B cells, veiled cells, interdigitating and follicular cells, splenocytes, thymocytes, microglia, Kupffer cells, endothelial cells, fibroblasts, eosinophils, and any cell displaying HLA-peptide complexes on its cell surface (U.S. Pat. No. 8,247,231). Agents capable of increasing the number of A1 adenosine receptors on the antigen-presenting cell plasma membrane include, but are not limited to, cisplatin, dexamethasone, daunorubicin, doxorubicin, mitoxantrone, carbamazepine, adenosine receptor antagonists, nucleotide sequences encoding the A1 adenosine receptor, for example, cDNA encoding the human A₁ adenosine receptor, allosteric enhancers, such as PD 81,723, which increases the affinity and binding of an A1 adenosine receptor ligand for A1 adenosine receptors and coupling of the receptor to the G protein; contacting the cells with divalent cations, including magnesium and calcium; and/or contacting the cells with adenosine deaminase, or immunomodulators or priming agents, such as lymphokines, MDP, MTP, MTP-PE, IFN-γ, PMA, GM-CSF, fMLP, or FLT3 ligand, and protein kinase inhibitors.

The number of A1 adenosine receptors may also be increased by subjecting the cells to ischemic conditions.

Drugs that stimulate an antitumor response include checkpoint stimulators, including CD 28, tumor necrosis factor receptor family including glucocorticoid-induced tumor necrosis factor receptor (GITR), OX40, and 4-1BB (CD137), CD40, stimulator of interferon genes (STING) agonists, and cytokines, including interferons (IFNs), including INF-α and INF-β, toll receptor agonists, IL-2, IL-15, IL-17, IL-21, and IL-7. Strategies that produce antitumor responses include oncolytic virus therapies, adoptive T cell therapy, wherein T cells are artificially enriched with tumor specific antigens, CAR T cell therapies, T cell receptor (TCR) therapies, and DNA-based, RNA-based and mRNA-based vaccines.

### BRIEF SUMMARY OF THE INVENTION

The present invention relates to treatment of a cancer tumor using UV light delivered directly to a tumor via circular dichroism.

Accordingly, in one embodiment, there is a method of treating a cancer tumor in a mammal comprising:
a) selecting a UV light source;
b) filtering the UV light through a quarter phase plate; and
c) administering the filtered UV light directly to the cancer tumor in a mammal.

### BRIEF DESCRIPTION OF THE DRAWINGS

FIG. 1 is a depiction of the UV light passing through the quarter phase plate (90 degrees) of the present invention.

FIG. 2 depicts the method of treating a cancer tumor of the present invention.

### DETAILED DESCRIPTION OF THE INVENTION

While this invention is susceptible to embodiment in many different forms, there is shown in the drawings, and will herein be described in detail, specific embodiments with the understanding that the present disclosure of such embodiments is to be considered as an example of the principles and not intended to limit the invention to the specific embodiments shown and described. In the description below, like reference numerals are used to describe the same, similar, or corresponding parts in the several views of the drawings. This detailed description defines the meaning of the terms used herein and specifically describes embodiments in order for those skilled in the art to practice the invention.

### Definitions

The terms “about” and “essentially” mean ±10 percent.

The terms “a” or “an”, as used herein, are defined as one or as more than one. The term “plurality”, as used herein, is defined as two or as more than two. The term “another”, as used herein, is defined as at least a second or more. The terms “including” and/or “having”, as used herein, are defined as comprising (i.e., open language). The term “coupled”, as used herein, is defined as connected, although not necessarily directly, and not necessarily mechanically.

The term “comprising” is not intended to limit inventions to only claiming the present invention with such comprising language. Any invention using the term comprising could be separated into one or more claims using “consisting” or “consisting of” claim language and is so intended.

Reference throughout this document to “one embodiment”, “certain embodiments”, “an embodiment”, or similar terms means that a particular feature, structure, or characteristic described in connection with the embodiment is included in at least one embodiment of the present invention. Thus, the appearances of such phrases in various places throughout this specification are not necessarily all referring to the same embodiment. Furthermore, the particular features, structures, or characteristics may be combined in any suitable manner in one or more embodiments without limitation.

The term “or”, as used herein, is to be interpreted as an inclusive or meaning any one or any combination. Therefore, “A, B, or C” means any of the following: “A; B; C; A and B; A and C; B and C; A, B, and C”. An exception to this definition will occur only when a combination of elements, functions, steps, or acts are in some way inherently mutually exclusive.

The drawings featured in the figures are for the purpose of illustrating certain convenient embodiments of the present invention and are not to be considered as limitation thereto. The term “means” preceding a present participle of an operation indicates a desired function for which there is one or more embodiments, i.e., one or more methods, devices, or apparatuses for achieving the desired function and that one skilled in the art could select from these or their equivalent in view of the disclosure herein, and use of the term “means” is not intended to be limiting.

As used herein, the term “cancer tumor or cancer” refers to the presence of cells with typical oncogenic cell characteristics (e.g., uncontrolled proliferation, loss of specialized function, immobility, significant metastatic potential, significant increase in anti-apoptotic activity, rapid growth and proliferation rates, and certain characteristic morphologies and cell markers). In some cases, the cancer cells will be in the form of a tumor; such cells may be present locally within the mammal or circulate in the bloodstream as independent cells, such as leukemia cells.

As used herein, “tumor” refers to all neoplastic cell growth and proliferation, whether malignant or benign, as well as all pre-cancerous and cancerous cells and tissues.

As used herein, a “solid tumor” is an abnormal tissue mass that generally does not contain cysts or fluid areas. By way of non-limiting example, solid tumors can be in the brain, colon, breast, prostate, liver, kidney, lung, esophagus, head and neck, ovary, cervix, stomach, colon, rectum, bladder, uterus, testis, and pancreas. In some embodiments, the solid tumor regresses or its growth slows or stagnates (arrest) after treatment of the solid tumor with the methods disclosed herein. In other embodiments, the solid tumor is malignant. In some embodiments, the cancer comprises stage 0 (zero) cancer. In some embodiments, the cancer comprises a stage I cancer. In some embodiments, the cancer comprises a stage II cancer. In some embodiments, the cancer comprises a stage III cancer. In some embodiments, the cancer comprises a stage IV cancer. In some embodiments, the cancer is refractory and/or metastatic. For example, cancer may be refractory to monotherapy with radiotherapy, chemotherapy, or immunotherapy.

In particular embodiments, the cancer can be (but not limited to) a newly diagnosed, relapsed and/or refractory cancer selected from the group consisting of: nasopharyngeal carcinoma (celnospherygeal cancer), synovial carcinoma, hepatocellular carcinoma, renal carcinoma, connective tissue cancer, melanoma, lung cancer, colorectal cancer, colon cancer, rectal cancer, colorectal cancer, brain cancer, laryngeal cancer, oral cancer, liver cancer, bone cancer, pancreatic cancer, choriocarcinoma, gastrinoma, pheochromocytoma, prolactinoma, T-cell leukemia/lymphoma, neuroma, von Hippel-Lindau disease, Zollinger-Ellison syndrome, adrenal cancer, anal cancer, bile duct cancer, bladder cancer, ureter cancer, brain cancer, oligodendroglioma, neuroblastoma, meningioma, spinal cord tumor, bone cancer, osteochondrosoma, chondrosarcoma, Ewing's sarcoma, primary site-unknown cancer, carcinoid cancer, gastrointestinal carcinoid cancer, fibrosarcoma, breast cancer, Paget's disease, cervical cancer, colorectal cancer, rectal cancer, esophageal cancer, gallbladder cancer, head cancer, eye cancer, neck cancer, kidney cancer, Wilms' tumor, liver cancer, Kaposi's sarcoma, prostate cancer, lung cancer, testicular cancer, Hodgkin's disease, non-Hodgkin's lymphoma, oral cancer, skin cancer, mesothelioma, multiple myeloma, ovarian cancer, endocrine pancreatic cancer, glucagon tumor, pancreatic cancer, parathyroid cancer, penile cancer, pituitary cancer, soft tissue sarcoma, retinoblastoma, small intestine cancer, stomach cancer, thymus gland cancer, thyroid cancer, trophoblastic cell cancer, hydatidiform mole, uterine cancer, endometrial cancer, vaginal cancer, vulvar cancer, acoustic neuroma, mycosis fungoides, pancreatic islet tumors, carcinoid syndrome, somatostatin tumors, gingival cancer, heart cancer, lip cancer, meningeal cancer, mouth cancer, nerve cancer, palate cancer, parotid gland cancer, peritoneal cancer, pharyngeal cancer, pleural cancer, salivary gland cancer, tongue cancer, and tonsil cancer.

As used herein, the term “treating” or the like refers to alleviating or ameliorating a disease or condition and/or symptoms associated therewith. It is to be understood that, although not excluded, treating a disease or condition does not require that the disorder, condition, or symptoms associated therewith be completely eliminated.

As used herein, the term “UV light source” refers to electromagnetic radiation with a wavelength shorter than that of visible light that is of from about 10 nm to about 400 nm in range. In some embodiments, sub-ranges of ultraviolet light may be used, and/or may overlap with light in the visible range, for example from about 100 to about 420 nm, or from about 200 to about 420 nm, or from about 300 to about 420 nm, or from about 350 to about 420 nm. When it has multiple optical frequencies, it is called polychromatic and if it has a single frequency, it is called monochromatic. In some cases, polychromatic light has a mixture of some number of discrete wavelength components, while in other cases its optical spectrum is continuous.

As used herein the term “quarter plate” or “quarter phase plate” refers to a wave plate, wherein when UV light is passed through the filter it creates a 90-degree phase difference. It is an optical device that alters the polarization state of a light wave travelling through it. The quarter-wave plate, converts linearly polarized light into circularly polarized light.

As used herein, the term “administering” refers to the physical introduction of the filtered UV light of the present invention to the cancer tumor. The administration is direct to the cancer tumor. So internal cancers can be treated with an endoscope or the like or directly shown on an exterior cancer, such as a skin cancer. In general, any device that provides a UV light directly to a cancer tumor is anticipated.

### Drawings

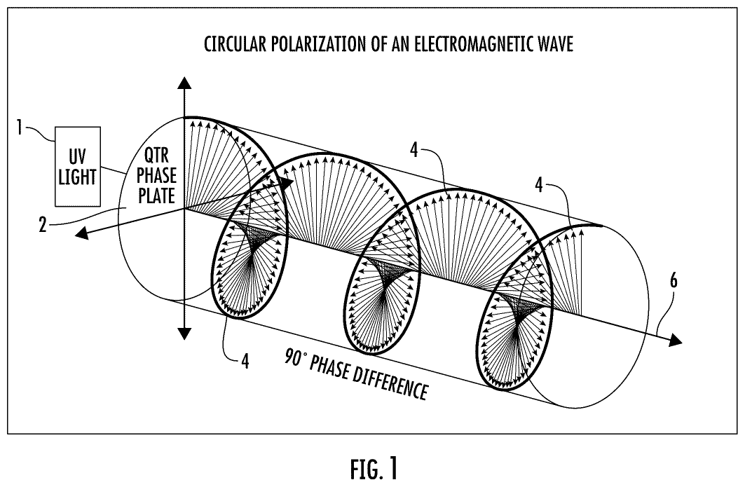

Now referring to the drawings, FIG. 1 is a depiction of the circular polarization of the UV light. UV light source 1 passes through quarter phase plate 2 creating a vector that would appear to be rotating as shown by the electric vectors 4 creating a 90-degree phase difference. The light then propagates in a straight line 6 where it can be utilized to treat a cancer tumor.

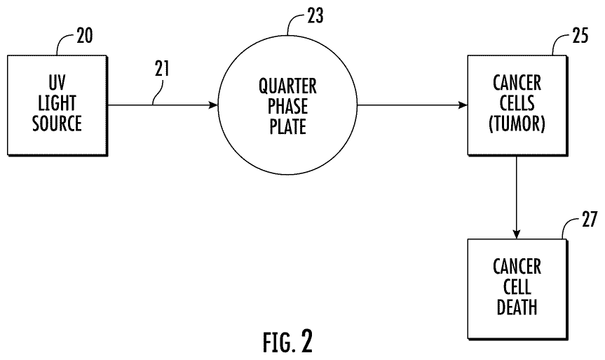

FIG. 2 is a depiction of the method of the present invention. UV light source 20 shines 21 through quarter phase plate 23. The light is then administered to the cancer tumor 25 causing cell death of cancer cells 27.

Those skilled in the art to which the present invention pertains may make modifications resulting in other embodiments employing principles of the present invention without departing from its spirit or characteristics, particularly upon considering the foregoing teachings. Accordingly, the described embodiments are to be considered in all respects only as illustrative, and not restrictive, and the scope of the present invention is, therefore, indicated by the appended claims rather than by the foregoing description or drawings. Consequently, while the present invention has been described with reference to particular embodiments, modifications of structure, sequence, materials, and the like apparent to those skilled in the art still fall within the scope of the invention as claimed by the applicant.

What is claimed is:

1. A method of treating a cancer tumor in a mammal comprising:
a) selecting a UV light source;
b) filtering the UV light through a quarter phase plate; and
c) administering the filtered UV light directly to the cancer tumor in a mammal.

2. The method of treating a cancer tumor in a mammal according to claim 1 wherein the UV light source is a polychromatic UV light.

3. The method of treating a cancer tumor in a mammal according to claim 1 wherein the UV light source has a wave length of from about 100 nm to about 400 nm.
